(19) United States
(12) Patent Application Publication
(10) Pub. No.: US 2005/0090836 A1
(43) Pub. Date: Apr. 28, 2005

(54) Umbilical cord care kit

(76) Inventor: Angela D. Stracener, Pitkin, LA (US)

Correspondence Address: MALABIKA J. GHOSH, 638 E. OLIVE AVE, #1, SUNNYVALE, CA, 94086, US

(21) Appl. No.: 10/946,501

(22) Filed: Sep. 21, 2004

Related U.S. Application Data
(60) Provisional application No. 60/504,193, filed on Sep. 22, 2003.

(51) Int. Cl. A61D 1/10; A61B 17/42; A61B 17/46

(52) U.S. Cl. 606/119; 606/120

(57) ABSTRACT

The present invention discloses a care kit for an umbilical cord for a newborn child. The care kit comprises a supply of materials necessary to care for an umbilical cord until it falls off which is estimated to be about two weeks. The present invention includes a wet umbilical cord cleaner, which have been wet with a disinfectant, and a dry umbilical cord cleaner to clean off any excess alcohol. Also included are clear adhesive dressings which are used during sponge baths to keep the umbilical cord dry. Additionally provided, are a convenient carrying case along with instructions for the umbilical cord care to prepare the parent for reporting back to the baby's doctor.

This application claims benefit of a prior U.S. provisional application Ser. No. 60/504193 filed on Sep. 22, 2003; all of which are herein incorporated by reference in their entirety.

### BACKGROUND OF THE INVENTION

1. Field of the Invention

The present invention generally relates to medical devices, and, more particularly, is concerned with a care kit for an umbilical cord for a newborn child.

2. Description of the Prior Art

Care kits for umbilical cords have been described in the prior art; however, none of the prior art devices disclose the unique features of the present invention.

In U.S. Pat. No. 5,817,103 dated Oct. 6, 1998, Bell disclosed an obstetrical combined disinfectant, clamp, cutter and containment system for umbilical cords, being housed in a comprehensive unit having increased efficiency in the area of motion saving and exposure to infectious organisms during the birthing process and when exposure to infectious organisms is undesirable. A housing contains three umbilical clamps as part of the overall housing of the unit and two pair of serrated, cutting blades incorporated into the housing by way of bonding to the housing structure of the unit. After the baby's umbilical cord is placed horizontally in the unit, the disinfectant makes contact with the cutting sites of the umbilical cord, followed by the closing of the unit, simultaneously clamping and cutting the cord, hereafter the unit is twisted at the perforated midpoint, separating the fetal umbilical stump from the maternal end of the umbilical cord. Following separation, both maternal and infant cord ends remain in a clean environment, being totally enclosed tin individual compartments. The maternal portion of the device is easily discarded after expulsion of the placenta. The infant's unit will remain in place from five to seven (5-7) days, at which point the cord gradually atrophies and the unit falls off.

Patent application Ser. Nos. 2003/0036733A1 and 2003/0195479 A1 describe an umbilical cord care apparatus and method of use wherein the invention involves assembling an injector together with applicators, filling the injector with cleansing solvent, cleaning the umbilical cord stump and wiping the cord with the wettable applicator.

While the medical device claimed in U.S. Pat. No. 5,817,103 for umbilical care kits may be suitable for the purposes for which they were designed, they would not be as suitable for the purposes of the present invention. The invention of the present application, as hereinafter described, is simple, effective and requires no assembly of the apparatus as compares with those described in patent application Ser. Nos. 2003/0036733 A1 and 2003/0195479 A1.

### SUMMARY OF THE PRESENT INVENTION

The present invention discloses a care kit for an umbilical cord for a newborn child. The care kit comprises a supply of materials necessary to care for an umbilical cord until it falls off which is estimated to be about two weeks. The present invention includes a wet umbilical cord cleaner, which have been wet with a disinfectant, and a dry umbilical cord cleaner to clean off any excess alcohol. Also included are clear adhesive dressings which are used during sponge baths to keep the umbilical cord dry. Additionally provided, are a convenient carrying case along with instructions for the umbilical cord care to prepare the parent for reporting back to the baby's doctor.

An object of the present invention is to care for the umbilical cord of a newborn child until the umbilical cord falls off. A further object of the present invention is to provide the necessary materials for a parent to care for the umbilical cord of a newborn child. A further object of the present invention is to provide materials necessary to report to the child's doctor in order to update the doctor so that he can provide the proper medical care to the child.

The foregoing and other objects and advantages will appear from the description to follow. In the description reference is made to the accompanying drawings, which form a part hereof, and in which is shown by way of illustration specific embodiments in which the invention may be practiced. These embodiments will be described in sufficient detail to enable those skilled in the art to practice the invention, and it is to be understood that other embodiments may be utilized and that structural changes may be made without departing from the scope of the invention. In the accompanying drawings, like reference characters designate the same or similar parts throughout the several views.

### BRIEF DESCRIPTION OF THE DRAWINGS

In order that the invention may be more fully understood, it will now be described, by way of example, with reference to the accompanying drawings in which:

FIG. 1 is a perspective view of parts of the present invention.

FIG. 2 is a perspective view of parts of the present invention.

FIG. 3 is an elevation view of parts of the present invention.

FIG. 4 is an elevation view of parts of the present invention.

FIG. 5 is a perspective view of parts of the present invention.

FIG. 6 is an exploded view of the present invention.

### LIST OF REFERENCE NUMERALS

With regard to reference numerals used, the following numbering is used throughout the drawings.

10 present invention

12 wet cleaner

14 cotton tip

16 stick

18 dry cleaner

20 cotton tip

22 stick

24 package for cleaners

26 wet cleaner pouch

28 dry cleaner pouch

30 tear-away line

32 dressing

34 peel-away adhesive cover

36 case

38 lid for case

### DETAILED DESCRIPTION OF THE PREFERRED EMBODIMENT

The following discussion describes in detail one embodiment of the present invention. This discussion should not be construed, however, as limiting the invention to those particular embodiments since practitioners skilled in the art will recognize numerous other embodiments as well.

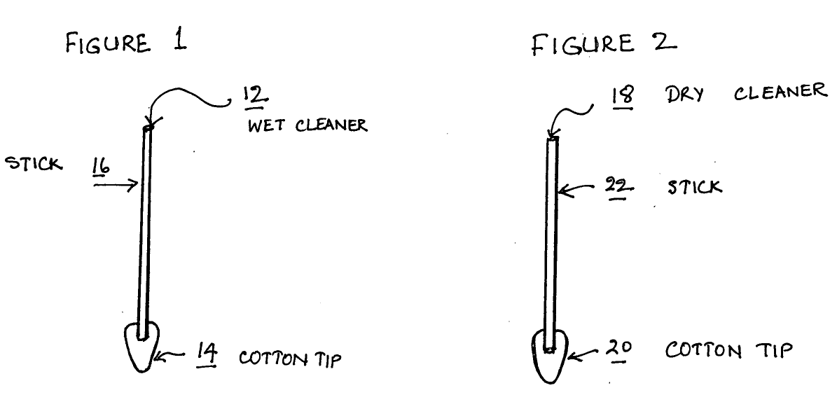

Turning to FIG. 1, shown therein is an umbilical cord cleaner 12, which has a cotton end 14, along with a plastic stick end 16 upon which the cotton tip is attached. The shape of the cotton tip can be any shape suitable for cleaning and disinfecting the umbilical cord stump including but not limited to oval, round, trapezoid or rectangular. The plastic stick 16 is intended to be held in the fingers of the user. The cotton tip 14 is saturated with alcohol such as, for example, isopropyl alcohol (70% v/v) or the like as a disinfectant.

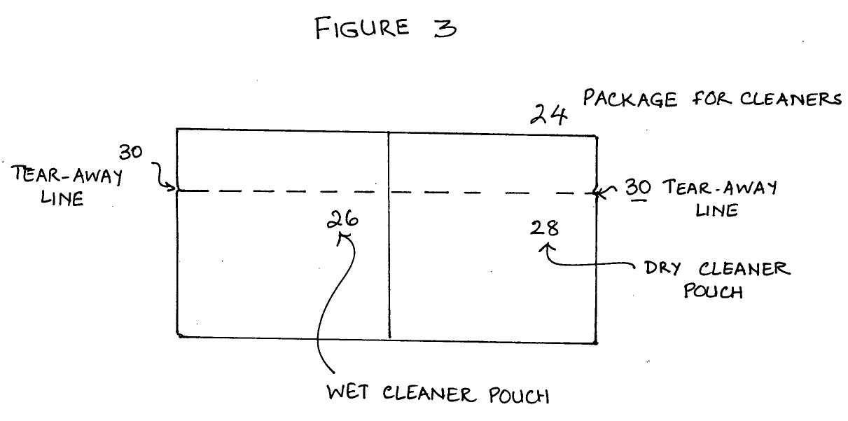

Turning to FIG. 2, therein is shown the dry cord cleaner 18 having a cotton tip end 20, along with a plastic stick end 22 upon which the cotton tip is disposed. The shape of the cotton tip can be any shape suitable for cleaning and disinfecting the umbilical cord stump including but not limited to oval, round, trapezoid, or rectangular. The dry cord cleaner 18 has no alcohol or other disinfectant saturated therein.

Turning to FIG. 3, therein is shown the packaging 24 for holding the cleaners 12, 18. Shown is the package 24 with a pouch portion for the wet cleaner 26 and a pouch portion for the dry cleaner 28, both of which have a tear-away line 30 shown therein so that a user can easily open the package. The packaging 24 is a sterile packaging having paper foil-lined inner pockets that tear away exposing the plastic cleaners 12, 18 so that the user can grasp the cleaners, pull them out and use them for cleaning.

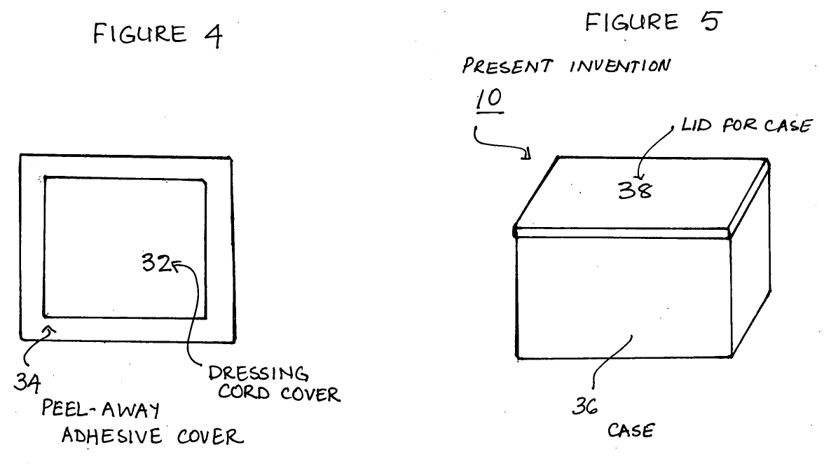

Turning to FIG. 4, therein is shown the dressing 32 for covering the umbilical cord while the infant is given a sponge bath. The shape and size of the dressing is any that is suitable for covering the umbilical cord stump and the area around it including but not limited to round, oval, or square. The dressing 32 has a peel-away adhesive covering 34 around its edges so that the clear dressing can be easily attached to the body of the baby. The covering 34 has a mild, adhesive coating which is designed for use on babies and yet keeps the dressing attached to the body of the baby so that the umbilical cord remains dry during the sponge bath.

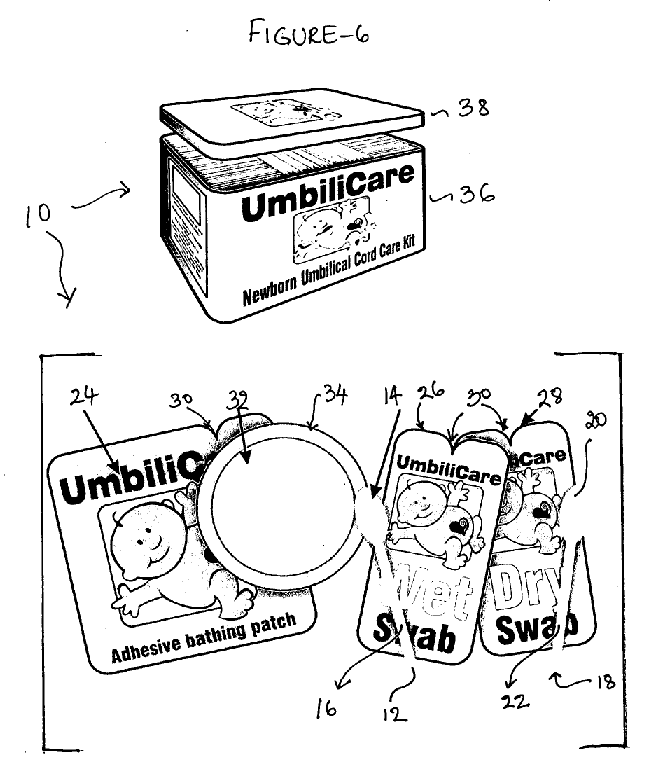

Turning to FIG. 5, therein is shown a small carrying case 36 which is used to contain the present invention 10 in its entirety. The carrying case 36 maybe made of metal or plastic and is designed so that it can be placed in a diaper bag to provide easy access and use with each diaper change. Furthermore, the carrying case has a lid 38 which snaps closed or is otherwise attached to the top of the case 36. The instructions for cord care are printed on the box for quick reference.

Turning to FIG. 6, therein is shown a case 36 which is shown as a box for containing the components of the present invention. Also shown are foil package 24 for containing the wet cleaner pouch 26 with wet swab 14, long with dry cleaner pouch 28 with dry swab 18. Also shown is the dressing 32.

In use, the umbilical cord care kit of the present invention 10 has an average of 14 days of supplies which is calculated by considering that newborns use approximately 10 to 12 diapers a day and that cord care is done with each diaper change which means 10 uses for 14 days which yields 140 cleaner sets. Furthermore, 14 clear dressings for a sponge bathing once per day are included. Also note that cord care is performed with each diaper change and after each bath.

1. An umbilical cord care kit comprising:
a container means for carrying the care kit;
one or more wet cleansing means;
one or more dry cleansing means;
one or more umbilical cord covers; and
a lid for closing the container means.

2. The kit of claim 1, wherein the wet cleansing means and the dry cleansing means are disposable applicators with cotton tips attached to plastic sticks and stored in individual sterile pouches.

3. The kit of claim 2, wherein the shape of the disposable applicators is oval, conical, rectangular or trapezoid.

4. The kit of claim 2, wherein the individual sterile pouches containing the wet and the dry cleansing means have a tear-away line to ease the opening of the pouches exposing the stick ends of the cleansing means.

5. The kit of claim 1, wherein the wet cleansing means is saturated with a disinfectant suitable for cleaning, disinfecting, sterilizing the umbilical cord stump and the areas surrounding the stump.

6. The kit of claim 5, wherein the disinfectant used to saturate the wet cleaning means is 70% v/v isopropyl alcohol.

7. The kit of the claim 2, wherein the sterile pouches containing the wet and the dry cleansing means are foil-lined.

8. The kit of claim 1, wherein the umbilical cord cover is a round, oval or square dressing that covers the umbilical cord stump.

9. The kit of claim 1, wherein the umbilical cord cover is clear.

10. The kit of claim 8, wherein the umbilical cord cover has a mild adhesive coating at the edges with detachable or peel-away covering which when removed allows the dressing to adhere to skin.

11. The kit of claim 1, wherein the wet and dry cleansing means are individually stored in sterile pouches together with the umbilical cord cover in a foil-lined sterile pouch.

12. The kit of claim 11, wherein the pouch containing the cord cover and the wet and dry cleansing means has a tear-away line to ease the opening of the pouch.

13. The kit of claim 1, wherein the foil-lined package containing the umbilical cord cover and the wet and the dry cleansing means are stored in a suitable container means which has a lid attached to the top of the container means.

14. The kit of claim 1, where in the container means is closed with a snap-close detachable lid.

15. The kit of claim 1, wherein the container means is made of metal or plastic.

16. The kit of claim 1, wherein the instructions for cord care are printed on the container for quick reference.

17. The kit of claim 1, wherein the container means comprises 140 wet and cleansing means each and 14 umbilical cord covers for umbilical cord care for 14 days.

18. The kit of claim 1, wherein the umbilical cord care kit is suitable for carrying the diaper bag.
